(19) United States
(12) Patent Application Publication
(10) Pub. No.: US 2024/0196889 A1
(43) Pub. Date: Jun. 20, 2024

(54) USE OF DI- AND TRICARBOXYLIC FATTY ACIDS IN THE PREVENTION OF BIOFILM FORMATION

(71) Applicant: Katholieke Universiteit Leuven, Leuven (BE)

(72) Inventors: Bram Lories, Halle (BE); Hans Steenackers, Leuven (BE)

(21) Appl. No.: 17/924,296

(22) PCT Filed: May 11, 2021
(86) PCT No.: PCT/EP2021/062469

§ 371 Date: Nov. 9, 2022

(30) Foreign Application Priority Data
May 11, 2020 (EP) 20173892.9

(51) Int. Cl. A01N 37/02; A01P 1/00; A61K 31/20; A61P 31/04

(52) U.S. Cl. CPC A01N 37/02; A01P 1/00; A61K 31/20; A61P 31/04

(57) ABSTRACT

The invention relates to the in vitro use of a di- or tri carboxylic acid with a chain length of between 15 and 23 carbon atoms, such as agaric acids in preventing formation, decreasing formation or delaying formation of a biofilm by bacteria on abiotic surfaces.

### FIELD OF THE INVENTION

The invention relates to the prevention and treatment of biofilms.

### BACKGROUND OF THE INVENTION

Bacteria typically live in dense communities encapsulated by a self-produced matrix, commonly known as biofilms. These biofilms are highly tolerant to antibiotics, disinfectants and mechanical removal, giving rise to chronic infections or contaminations [Costerton et al. (1999) Science 284, 1318-1322; Fux et al. (2005) Trends in microbiology 13, 34-40; Hall-Stoodley and Stoodley (2009) Cellular microbiology 11, 1034-1043] The highly tolerant and persistent nature of biofilms causes enormous problems in a wide variety of sectors, including medicine, food industry and agriculture [Galié et al. (2018) Frontiers in microbiology 9, 898-898; Velmourougane et al. (2017) Journal of Basic Microbiology 57, 548-573; Koo et al. (2017) Nature reviews. Microbiology 15, 740-755]. The failure of current strategies to completely prevent or remove biofilms invokes a strong need for novel biofilm inhibitors. Preventive strategies that block initial adhesion seem most promising because of the low permeability of yet established biofilms [Roy et al. (2018) Virulence 9, 522-554].

One biofilm forming pathogen that is particularly problematic in the food and feed industry is Salmonella. Globally, there are an approximate 94 million cases of Salmonella annually, leading to 155 000 deaths [Roy et al. (2018) Virulence 9, 522-554]. More than 85% of these cases are estimated to be foodborne, making Salmonella the most common cause of bacterial foodborne outbreaks. In 2017, the European food safety agency reported over 90 000 cases of illness due to Salmonella, resulting in 156 deaths [EFSA (2018) EFSA Journal 2018; 16(12), 5500].

In an ongoing screening for novel anti-biofilm compounds, we identified agaric acid as a potent Salmonella biofilm inhibitor. Agaric acid or 2-hydroxynonadecane-1,2,3-tricarboxylic acid is a fatty acid naturally produced by certain fungi. It been reported as an inhibitor of the mitochondrial adenine nucleotide exchange reaction that induces mitochondrial permeability [García et al. (2005) Mitochondrion 5, 272-281]. Historically, this compound has been used as an anhidrotic to symptomatically treat extreme sweating in tuberculosis patients [García et al. (2005) Mitochondrion 5, 272-281]. Additionally, at high dosages, agaric acid can inhibit the nervous, respiratory, and circulatory systems in lower animals. Therefore, agaric acid has also been utilized as a metabolic inhibitor in animal experiments [Freedland & Newton (1981) Methods Enzymol. 72, 497-506].

Celleno et al. (2019) G Ital Dermatol Venereol. 154, 338-341 describes the use of Agaric acid as an antiperspirant for hyperhidrosis.

### SUMMARY OF THE INVENTION

The present invention show that agaric acid—when used in a preventive manner-significantly inhibits Salmonella biofilm formation: it significantly reduces the number of bacteria and the amount of biomass adhering to abiotic surfaces via downregulation of flagellar rotation genes and inhibition of swimming motility. Importantly, the reduced biofilm formation leads to more effective treatment with hydrogen peroxide, a common disinfectant in food industry.

Agaric acid or other di- and tri carboxylic acids can be included in as anti-biofilm agent in coatings, as well as in cleaning agents, or can be added in liquid circuits.

The present invention also relates to the use of agaric acid or other di- and tri carboxylic acids in the inhibiting the biofilm formation of mixed-species communities.

The present invention also relates to the use of agaric acid or other di- and tri carboxylic acids in weaken mixed-species biofilm communities and reduce the tolerance towards antimicrobials.

The invention is further summarised in the following statements:

1. In vitro use of a di- or tri carboxylic acid with a chain length of between 8 and 41 in treating, preventing formation, decreasing formation or delaying formation of a biofilm by micro-organisms on abiotic surfaces.

2. The use according to statement 1, wherein planktonic growth of the micro-organisms is preserved.

3. The use according to statement 1 or 2, wherein the micro-organisms are bacteria.

4. The use according to any one of statements 1 to 3, wherein the carboxylic acid is a tricarboxylic acid.

5. The use according to any one of statements 1 to 4, wherein the carboxylic acid is substituted with an hydroxyl group.

6. The use according to any one of statements 1 to 5, wherein the carboxylic acid is a 1,2,3 tricarboxylic acid.

7. The use according to any one of statements 1 to 6, wherein the carboxylic acid is a 2-OH, 1,2,3 tricarboxylic acid.

8. The use according to any one of statements 1 to 7, wherein the carboxylic acid has a C chain of between 15 and 23 carbon atoms.

9. The use according to any one of statements 1 to 8, wherein the carboxylic acid has a C chain of between 17 and 21 carbon atoms.

10. The use according to any one of statements 1 to 9, wherein the carboxylic acid is selected from the group consisting of 2-hydroxyheptadecane-1,2,3-tricarboxylic acid-(C₂₀H₃₆O₇); 2-hydroxynonadecane-1,2,3-tricarboxylic acid (Agaric acid) (C₂₂H₄₀O₇); 2-hydroxyoctadecane-1,2,3-tricarboxylic acid (C₂₁H₃₈O₇); 2-Hydroxy-4-oxononadecane-1,2,3-tricarboxylic acid (C₂₂H₃₈O₈) and 2-Hydroxy-19-methylicosane-1,2,3-tricarboxylic acid (C₂₄H₄₄O₇).

11. The use according to any one of statements 1 to 10, wherein the carboxylic acid is 2-hydroxynonadecane-1,2,3-tricarboxylic acid (agaric acid).

12. The use according to any one of statements 1 to 11, wherein the carboxylic acid is used at a concentration of at least 5 μM, at least 10 μM, at least 25 μM, at least 50 μM, at least 100 μM, or at least 500 μM.

13. The use according to any one of statements 1 to 12, in the prevention of Salmonella Typhimurium biofilm formation, S. aureus biofilm formation or E. coli biofilm formation.

14. The use according to any one of statements 1 to 13, followed by the use of an antimicrobial, typically antibacterial, agent. Such antimicrobial against can kill planktonic bacteria, but can also be used against adherent bacteria which tend to form biofilm formation, or at are an initials stage of biofilm formation

15. The method according to statement 14, wherein the antimicrobial agent is an oxidizing agent, typically a peroxide, such as hydrogen peroxide.

16. A di- or tri carboxylic acid with a chain length of between C8 and C41 in the treatment or prevention of bacterial biofilm formation.

17. The carboxylic acid for use in the treatment or prevention in accordance with statement 16, wherein the use is a topical use.

18. The carboxylic acid for use in the treatment or prevention in accordance with statement 16 or 17, in preventing or treating biofilm formation on implants.

19. The carboxylic acid for use in the treatment or prevention in accordance with any one of statements 16 to 18, wherein the carboxylic acid is a tricarboxylic acid.

20. The carboxylic acid for use in the treatment or prevention in accordance with any one of statements 16 to 19, wherein the carboxylic acid is substituted with an hydroxyl group.

21. The carboxylic acid for use in the treatment or prevention in accordance with any one of statements 16 to 20, wherein the carboxylic acid is a 1,2,3 tricarboxylic acid.

22. The carboxylic acid for use in the treatment or prevention in accordance with any one of statements 16 to 21, wherein the carboxylic acid is a 2-OH 1,2,3 tricarboxylic acid.

23. The carboxylic acid for use in the treatment or prevention in accordance with any one of statements 16 to 22, wherein the carboxylic acid has a C chain of between 15 and 23 carbon atoms.

24. The carboxylic acid for use in the treatment or prevention in accordance with any one of statements 16 to 23, wherein the carboxylic acid has a C chain of between 17 and 21 carbon atoms.

25. The carboxylic acid for use in the treatment or prevention in accordance with any one of statements 16 to 24, wherein the carboxylic acid is selected from the group consisting of 2-hydroxyheptadecane-1,2,3-tricarboxylic acid-(C₂₀H₃₆O₇); 2-hydroxynonadecane-1,2,3-tricarboxylic acid (Agaric acid) (C₂₂H₄₀O₇); 2-hydroxyoctadecane-1,2,3-tricarboxylic acid (C₂₁H₃₈O₇); 2-Hydroxy-4-oxononadecane-1,2,3-tricarboxylic acid (C₂₂H₃₈O₈) and 2-Hydroxy-19-methylicosane-1,2,3-tricarboxylic acid (C₂₄H₄₄O₇).

26. The carboxylic acid for use in the treatment or prevention in accordance with any one of statements 16 to 25, wherein the carboxylic acid is 2-hydroxynonadecane-1,2,3-tricarboxylic acid (agaric acid).

### DETAILED DESCRIPTION

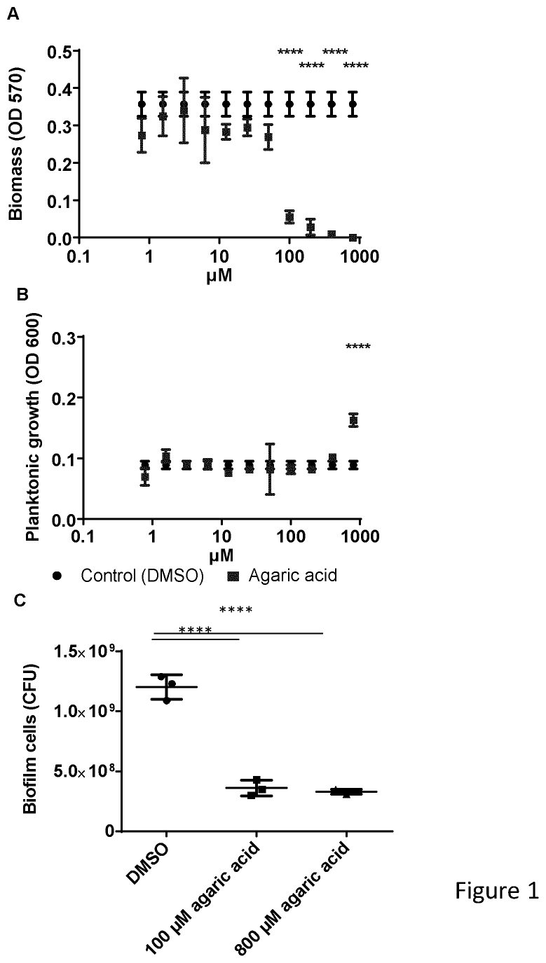

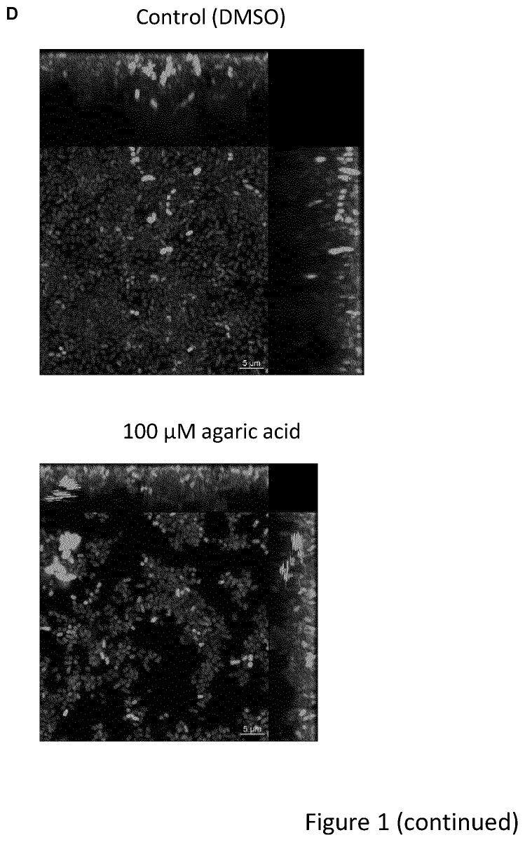

FIG. 1: Agaric acid has no bactericidal effect but strongly inhibits Salmonella biofilm formation.

A) Agaric acid inhibits biofilm formation of Salmonella Typhimurium ATCC 14028 in a concentration dependent manner as measured via crystal violet staining in the Calgary biofilm device. The mean and standard deviation of three biological repeats are shown. Significant differences were determined via a one-way ANOVA with Bonferroni multiple comparisons corrections. B) Agaric acid does not inhibit planktonic growth at concentrations relevant for biofilm inhibition. Planktonic growth was measured as the OD600 of the liquid culture after 48 h incubation in the Calgary biofilm device. The mean and standard deviation of three biological repeats are shown. Significant differences were determined via a one-way ANOVA with Bonferroni multiple comparisons corrections. C) Agaric acid also reduces the number of cells that adhere to the bottom of a glass petri dish. Significant differences were determined via a one-way ANOVA with Bonferroni multiple comparisons corrections. D) Maximum intensity projection top and side view of fluorescently labelled Salmonella biofilms shows that the presence of agaric acid results in less dense and scattered biofilms.

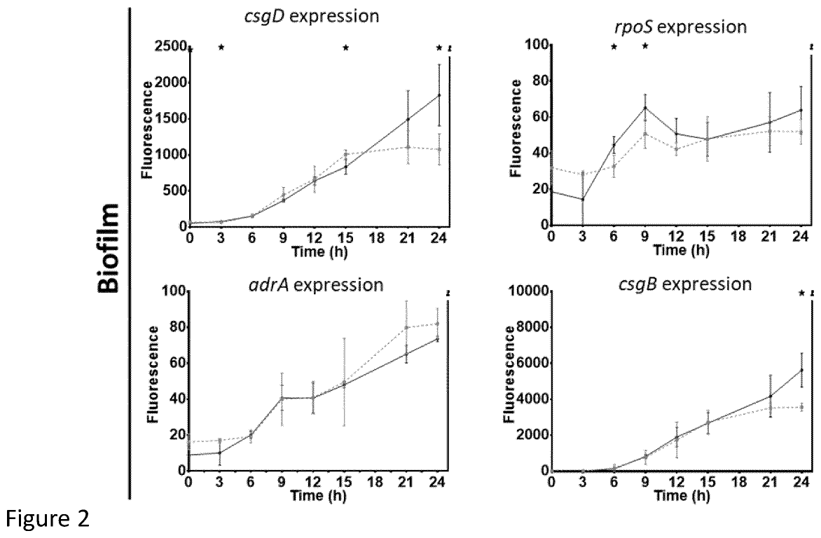

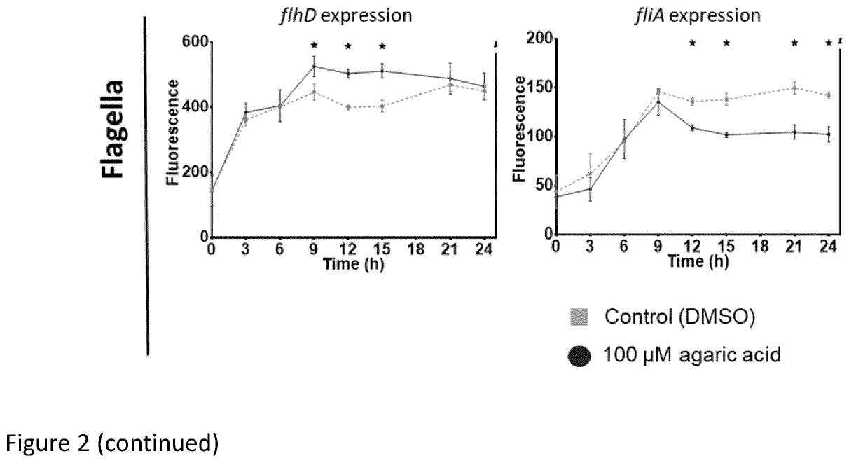

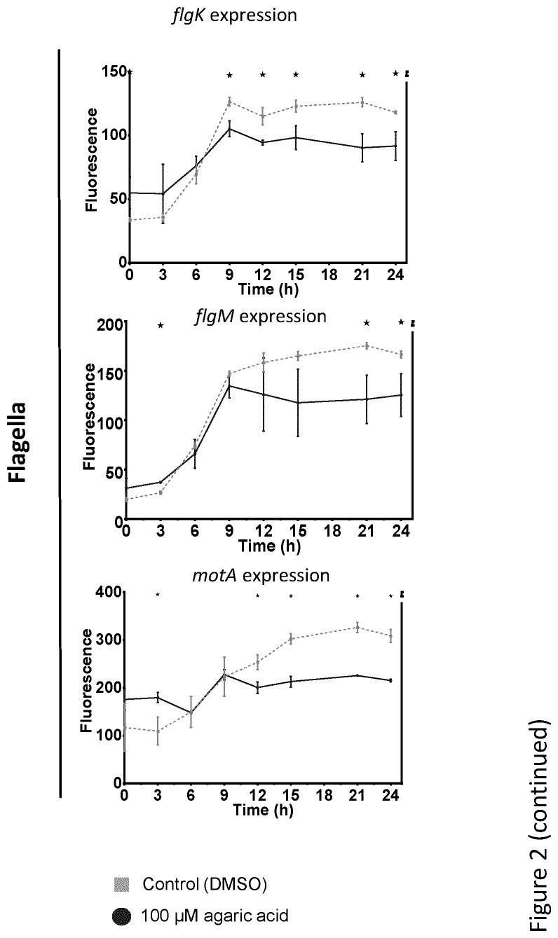

FIG. 2: Agaric acid downregulates transcription of flagella genes.

Fluorescence as a measure of gene transcription at different time points is shown for Salmonella Typhimurium ATCC 14028 grown in DMSO (squares, light line) or 100 μM agaric acid (circles, dark line). The mean and standard deviation of three biological repeats are depicted. Asterisks indicate significant differences as determined by a two-tailed Student t-test (P<0.05). Unexpectedly, genes important for biofilm formation were upregulated by agaric acid (panel A). Agaric acid did reduce the transcription of Class II and III flagella genes (panel B).

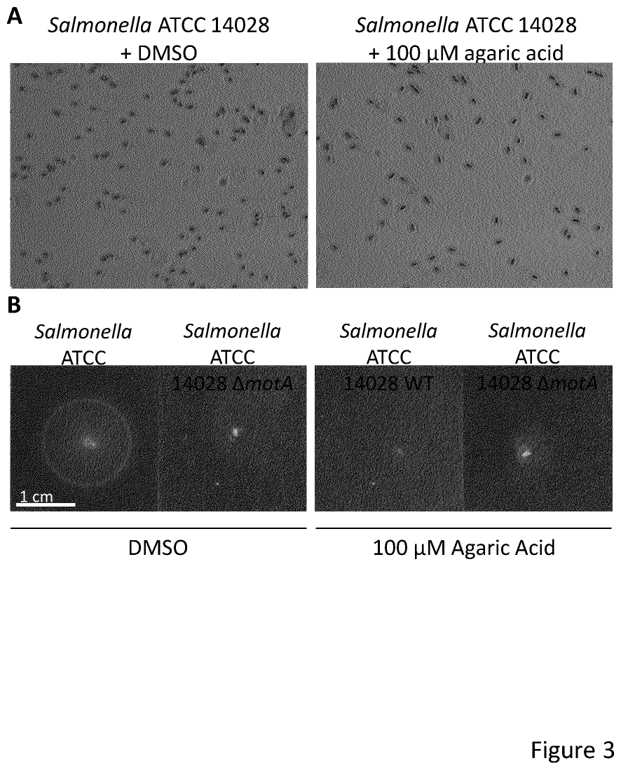

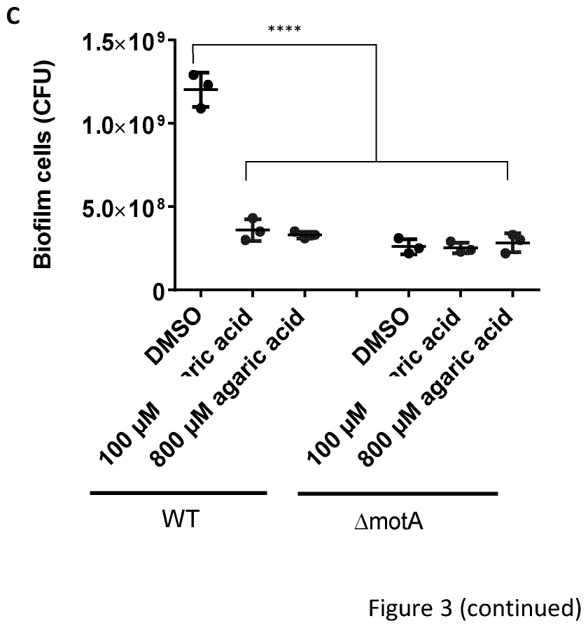

FIG. 3: Agaric acid reduces biofilm formation via inhibition of flagellar motility.

A) Phase contrast microscopy showed no difference between flagella of Salmonella grown in 100 μM agaric acid or corresponding amount of DMSO as visualised via a crystal violet based flagella staining. One representative repeat of three biological repeats is shown. B) 100 μM agaric acid almost completely inhibited motility in a soft agar swimming, similar to a motA deletion mutant. One representative repeat of three biological repeats is shown. C) Deletion of motA inhibited biofilm formation to a similar extent as a preventive treatment with agaric acid. Moreover, agaric acid does not further reduce biofilm formation of the motA deletion mutant. Three biological repeats are shown. Significant differences were determined via a one-way ANOVA with Bonferroni multiple comparisons corrections.

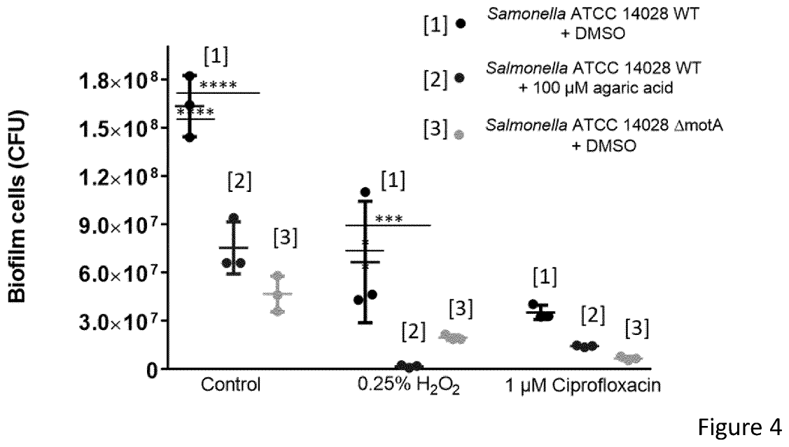

FIG. 4: Agaric increases susceptibility to treatment with H₂O₂. Biofilms, grown in presence of 100 μM agaric or the corresponding amount of DMSO, were treated with 0.25% H₂O₂ or 1 μM ciprofloxacin for 1 h. A motA deletion mutant was used as a control. Agaric acid significantly reduced the number of biofilm cells that survive treatment with 0.25% H₂O₂. Three biological repeats are shown. Significant differences were determined via a two-way ANOVA with Bonferroni multiple comparisons corrections.

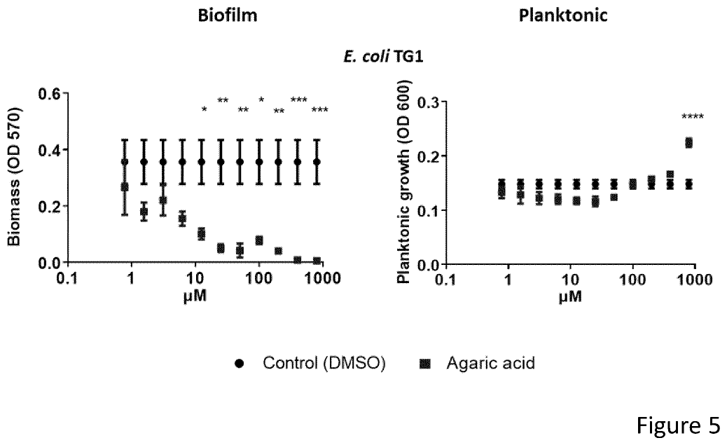

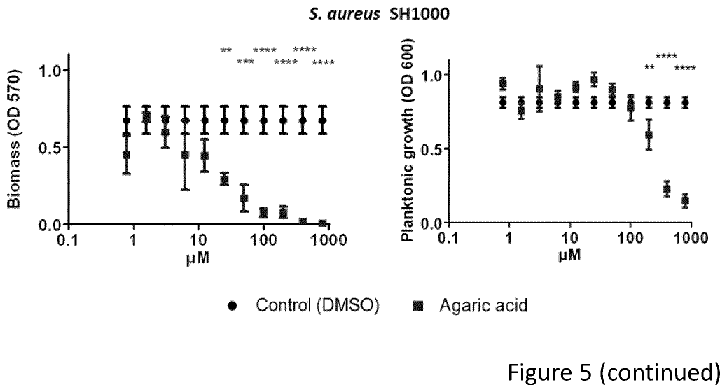

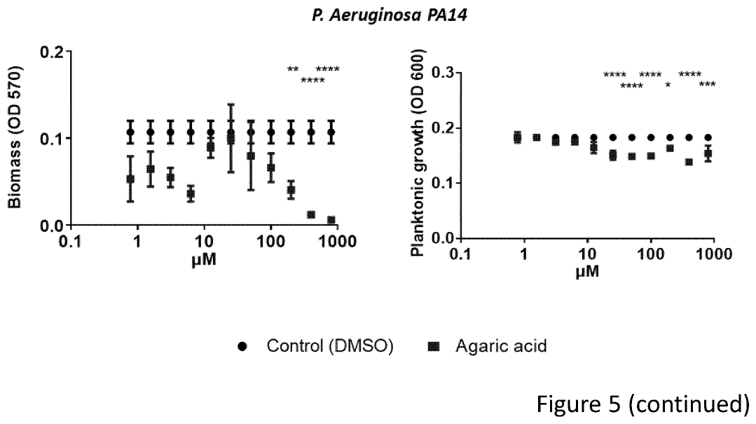

FIG. 5: Agaric acid also reduces biofilm formation of other pathogens.

Agaric acid inhibits biofilm formation of E. coli TG1, P. aeruginosa PA14 and S. aureus SH1000 as measured via crystal violet staining in the Calgary biofilm device. The effect on planktonic growth was measured via OD₆₀₀ measurement of the broth in the well. The mean and standard deviation of three biological repeats are shown. Significant differences were determined via a one-way ANOVA with Bonferroni multiple comparisons corrections.

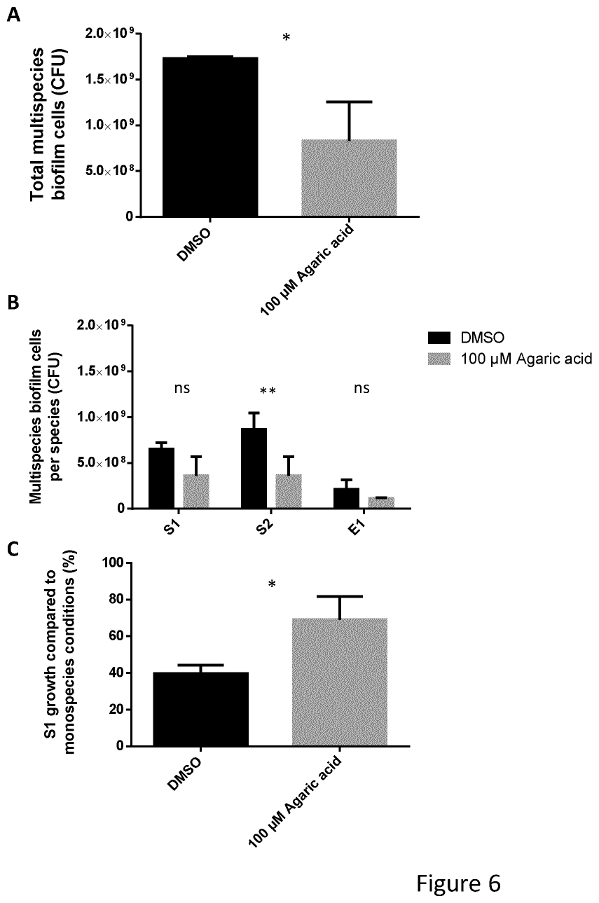

FIG. 6: Agaric acid reduces mixed-species biofilm formation. A) Agaric acid significantly reduces the biofilm formation of a mixed-species community consisting of two Salmonella and one E. coli strain. The mean and standard deviation of three biological repeats are depicted. Asterisks indicate significant differences as determined by a two-tailed Student t-test (P<0.05). B) Only the biofilm formation of the S2 strain is significantly inhibited by 100 μM agaric acid in the mixed-species community. The mean and standard deviation of three biological repeats are depicted. Significant differences were determined via a two-way ANOVA with Sidak's multiple comparisons corrections (P<0.05). C) Agaric acid weakens the competition experienced by S1 in presence of S2 and E1. The mean and standard deviation of three biological repeats are depicted. Significant differences were determined via a paired two-tailed Student t-test (P<0.05).

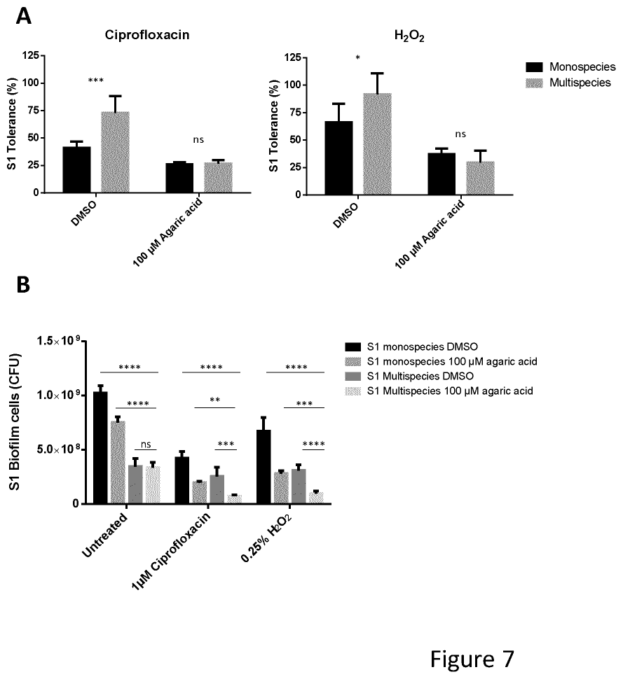

FIG. 7. Agaric acid inhibits the enhanced tolerance in mixed-species communities. A) Agaric acid abolishes the increased tolerance of S1 towards treatment with ciprofloxacin or H₂O₂ in mixed-species conditions. The mean and standard deviation of six biological repeats for the DMSO controls and three biological repeats for the agaric acid treated biofilms are depicted. Significant differences were determined via a two-way ANOVA with Sidak's multiple comparisons corrections (P<0.05). B) Mixed-species communities pretreated with agaric acid show the highest susceptibly towards antimicrobial treatment. The mean and standard deviation of six biological repeats for the DMSO controls and three biological repeats for the agaric acid treated biofilms are depicted. Significant differences were determined via a two-way ANOVA with Sidak's multiple comparisons corrections (P<0.05).

“Biofilm” in the context of the present invention relates to a syntrophic consortium of microorganisms in which cells stick to each other and often also to a surface. These adherent cells become embedded within a slimy extracellular matrix that is composed of extracellular polymeric substances (EPS). The cells within the biofilm produce the EPS components, which are typically a polymeric conglomeration of extracellular polysaccharides, proteins, lipids and DNA.

Typical examples of biofilm generating bacteria are Salmonella, Bacillus, Escherichia coli, Pseudomonas aeruginosa, Staphylococcus aureus and Listeria monocytogenes.

“Planktonic bacteria” refers to free-living bacteria residing in a liquid phase, not attached to a surface.

“Agaric acid” or “agaricin” refers to 2-hydroxynonadecane-1,2,3-tricarboxylic acid with chemical formula C₂₂H₄₀O₇. The structure of agaric acid is depicted in formula I below:

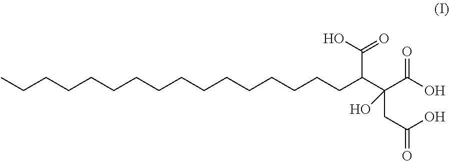 [SMILES: CCCCCCCCCCCCCCCCC(C(=O)O)C(O)(CC(=O)O)C(=O)O]

Agaric acid is a specific embodiment of a larger group of di- and tricarboxylic fatty acids.

With reference to the “carbon chain length” or “C-Chain”, the C atoms of the COOH groups or other C comprising substituents are not counted to determine the chain length. Thus agaric acid with chemical formula C₂₂H₄₀O₇ and three COOH groups has a chain length of 19.

In case of branched C chain, C-chain refers to longest branch of the C-chain.

Examples of di- or tricarboxylic fatty acids with branched C-chain are 5-Hydroxy-6-octyloctadecane-3,4,5-tricarboxylic acid or 3,5-Diethyl-4-hydroxyhenicosane-2,3,4-tricarboxylic acid.

Compounds for use in the methods of the present invention are di- and tricarboxylic fatty acids, in particular tricarboxylic fatty acids.

Numbering of substituents of di- and tricarboxylic fatty acids is in accordance with agaric acids wherein COOH groups are at position 1, 2 and 3 and OH is at position 2.

COOH groups of dicarboxylic fatty acids typically occur at positions 1 and 2, or positions 2 or 3.

COOH groups of tricarboxylic fatty acids typically occur at positions 1,2,3 or 1,3,4 or 2,3,4 or 3,4,5, most particular on position 1,2,3.

Di- and tricarboxylic fatty acids have a chain length of between 8 and 41 carbon atoms, more particular between 11 and 25 carbon atoms, 13 and 23 carbon atoms, 15 and 23 carbon atoms, 17 and 21 carbon atoms, or have a chain length 19 carbon atoms.

Typically di- and tricarboxylic fatty acids in the present invention are further substituted with one or more hydroxyl groups, typically one hydroxyl group at position 2 as shown in for example agaric acid.

Additional examples of further substituents are for example, CH₃, (CH₃)₂, C₂H₅, CH₃—CO, COOCH₃ COOC₄H₉COOC₅H₁₁, CH₃(CO₂)O, O.

The C-Chain in the di- and tricarboxylic fatty acids may be linear or branched.

Examples of compounds with a branched C chain are 4-Butan-2-yl-5-hydroxy-3,7-dimethylnonane-3,4,5-tricarboxylic acid, 5-Hydroxy-6-octyloctadecane-3,4,5-tricarboxylic acid or 3,5-Diethyl-4-hydroxyhenicosane-2,3,4-tricarboxylic acid.

Typically the C-chain is unsaturated.

Explicitly disclosed herein are di- and tricarboxylic fatty acids with any combination of the above mentioned features.

Specific compounds for use in the present invention are: 2-hydroxyheptadecane-1,2,3-tricarboxylic acid-(C₂₀H₃₆O₇); 2-hydroxy-2-(2-methoxy-2-oxoethyl)-3-tetradecylbutanedioic acid (C₂₁H₃₈O₇); 2-hydroxynonadecane-1,2,3-tricarboxylic acid (Agaric acid) C₂₂H₄₀O₇); 2-hydroxyoctadecane-1,2,3-tricarboxylic acid (C₂₁H₃₈O₇); 2-Hydroxy-2-(16-methylheptadecyl)butanedioic acid (C₂₂H₄₂O₅); 2-Hydroxy-4-oxononadecane-1,2,3-tricarboxylic acid (C₂₂H₃₈O₈); and 2-Hydroxy-19-methylicosane-1,2,3-tricarboxylic acid (C₂₄H₄₄O₇).

Another group of specific compounds for use in the present invention are 2-hydroxyheptadecane-1,2,3-tricarboxylic acid-(C20H3607); 2-hydroxynonadecane-1,2,3-tricarboxylic acid (Agaric acid) (C22H40O7); 2-hydroxyoctadecane-1,2,3-tricarboxylic acid (C21H38O7); 2-Hydroxy-4-oxononadecane-1,2,3-tricarboxylic acid (C22H38O8) and 2-Hydroxy-19-methylicosane-1,2,3-tricarboxylic acid (C24H44O7).

TABLE 1
examples of di- and tricarboxylic acids

| | Chemical | | | Other |
|---|---|---|---|---|
| Name | formula | C-chain | COOH | subsituents |
| 4-Acetyl-2-hydroxyoctane-1,2,3- | C₁₃H₂₀O₈ | 8 | 1, 2, 3 | OH, CH3-CO |
| tricarboxylic acid | | | | |
| 4-Butan-2-yl-5-hydroxy-3,7- | C₁₈H₃₂O₇ | 9 | 3, 4, 5 | OH, (CH3)2, |
| dimethylnonane-3,4,5- | | | | C4H9 |
| tricarboxylic acid | | | | |
| 2,4,4-Trihydroxyundecane- | C₁₄H₂₄O₉ | 11 | 1, 2, 3 | (OH)3 |
| 1,2,3-tricarboxylic acid | | | | |
| 2-Hydroxyundecane-1,2,3- | C₁₄H₂₄O₇ | 11 | 1, 2, 3 | OH |
| tricarboxylic acid | | | | |
| 2-(1-Butoxy-9-methyl-1- | C₁₉H₃₄O₇ | 11 | 1, 2 | OH, |
| oxodecan-2-yl)-2- | | | | COOC4H9, CH3 |
| hydroxybutanedioic acid | | | | |
| 2-hydroxytridecane-1,2,3- | C₁₆H₂₈O₇ | 13 | 1, 2, 3 | OH |
| tricarboxylic acid | | | | |
| 2-Dodecyl-2-hydroxybutanedioic | C₁₆H₃₀O₅ | 14 | 1, 2 | OH |
| acid | | | | |
| 2-hydroxytetradecane-1,2,3- | C₁₇H₃₀O₇ | 14 | 1, 2, 3 | OH |
| tricarboxylic acid | | | | |
| 3-Hydroxytetradecane-1,3,4- | C₁₇H₃₀O₇ | 14 | 1, 3, 4 | OH |
| tricarboxylic acid | | | | |
| 1,2,3-Trihydroxypentadecane- | C₁₈H₃₂O₉ | 15 | 1, 2, 3 | (OH)₃ |
| 1,2,3-tricarboxylic acid | | | | |
| 2-Hydroxypentadecane-1,2,3- | C₁₈H₃₂O₇ | 15 | 1, 2, 3 | OH |
| tricarboxylic acid | | | | |
| 3-Hexanoyl-2-hydroxy-14- | C₂₅H₄₄O₈ | 15 | 1, 2, 3 | OH, COOC₅H₁₁, |
| methylpentadecane-1,2,3- | | | | CH₃ |
| tricarboxylic acid | | | | |
| 2-Hexadecyl-2- | C₂₀H₃₈O₅ | 16 | 1, 2 | OH |
| hydroxybutanedioic acid | | | | |
| 2-Hydroxyhexadecane-1,2,3- | C₁₉H₃₄O₇ | 16 | 1, 2, 3 | OH |
| tricarboxylic acid | | | | |
| 2-hydroxyheptadecane-1,2,3- | C₂₀H₃₆O₇ | 17 | 1, 2, 3 | OH |
| tricarboxylic acid | | | | |
| 2-hydroxy-2-(2-methoxy-2- | C₂₁H₃₈O₇ | 17 | 2, 3 | OH, COOCH₃ |
| oxoethyl)-3- | | | | |
| tetradecylbutanedioic acid | | | | |
| 5-Hydroxy-6-octyloctadecane- | C₂₉H₅₄O₇ | 18 | 3, 4, 5 | OH, C₈H₁₇ |
| 3,4,5-tricarboxylic acid | | | | |
| 2-hydroxynonadecane -1,2,3- | C₂₂H₄₀O₇ | 19 | 1, 2, 3 | OH |
| tricarboxylic acid (Agaric acid) | | | | |
| 2-hydroxyoctadecane-1,2,3- | C₂₁H₃₈O₇ | 18 | 1, 2, 3 | OH |
| tricarboxylic acid | | | | |
| 2-Hydroxy-2-(16- | C₂₂H₄₂O₅ | 19 | 1, 2 | OH, CH₃ |
| methylheptadecyl)butanedioic | | | | |
| acid | | | | |
| 2-Hydroxy-4-oxononadecane- | C₂₂H₃₈O₈ | 19 | 1, 2, 3 | OH, O |
| 1,2,3-tricarboxylic acid | | | | |
| 2-Hydroxy-19-methylicosane- | C₂₄H₄₄O₇ | 20 | 1, 2, 3 | OH, CH₃ |
| 1,2,3-tricarboxylic acid | | | | |
| 3,5-Diethyl-4- | C₂₈H₅₂O₇ | 21 | 2, 3, 4 | OH, C₂H₅, C₂H₅ |
| hydroxyhenicosane-2,3,4- | | | | |
| tricarboxylic acid | | | | |
| 2-Hydroxy-2-icosylbutanedioic | C₂₄H₄₆O₅ | 22 | 1, 2 | OH |
| acid | | | | |
| 2-Docosyl-2-hydroxybutanedioic | C₂₆H₅₀O₅ | 24 | 1, 2 | OH |
| acid | | | | |
| 2-hydroxy-4-oxopentacosane- | C₂₈H₅₀O₈ | 25 | 1, 2, 3 | OH, O |
| 1,2,3-tricarboxylic acid | | | | |
| 2-(1-Acetyloxy-1- | C₃₁H₅₆O₈ | 27 | 1, 2 | OH, CH₃(CO₂)O |
| oxopentacosan-2-yl)-2- | | | | |
| hydroxybutanedioic acid | | | | |
| 2-hydroxytritriacontane-1,2,3- | C₃₆H₆₈O₇ | 33 | 1, 2, 3 | OH |
| tricarboxylic acid | | | | |
| 2-hydroxyhexatriacontane-1,2,3- | C₃₉H₇₄O₇ | 36 | 1, 2, 3 | OH |
| tricarboxylic acid | | | | |
| 2-Hydroxyhentetracontane- | C₄₄H₈₄O₇ | 41 | 1, 2, 3 | OH |
| 1,2,3-tricarboxylic acid | | | | |

“prevention” in the context of the present invention means that use of the compounds of the invention results in the formation of no biofilm at all, or a reduction in the amount of biofilm formation by 75, 80, 90 or 95% compared to a reference without use of a biofilm preventing compound.
“delay” in the context of the present invention means that the amount of biofilm obtained with a compound of the invention, is obtained 2, 5, 10, or 20 times slower compared to a reference without use of a biofilm preventing compound.

Agaric acid was identified as a novel inhibitor of Salmonella biofilms that does not reduce planktonic growth. This biofilm specific effect could be a major advantage as it has been hypothesized that there is no selection pressure for resistant mutants if virulence traits such as biofilms are targeted instead of growth [Allen et al. (2014) Nature Reviews Microbiology 12, 300]. Additionally, it has been suggested that biofilm-specific inhibitors could increase the risk that a contamination spreads as dispersion is enhanced [Fleming & Rumbaugh (2017) Microorganisms 5, 15]. However, this potential drawback is diminished in the case of agaric acid because flagellar motility is abrogated. Moreover, motility in itself is also an important virulence factor, further expanding the possible application fields of agaric acid [Josenhan & Suerbaum (2002) Int. J. of Medical Microbiology 291, 605-614]. Agaric acid thus shows strong potential for industrial and medical use.

### EXAMPLES

### Example 1. Agaric acid reduces Salmonella biofilm formation

A crystal violet based screening assay using the Calgary biofilm device revealed agaric acid as a potent inhibitor of Salmonella Typhimurium biofilm formation. Agaric acid significantly prevented biofilm formation at concentrations higher than 100 μM, reaching 99.9% inhibition at 800 μM (FIG. 1A). Crystal violet staining measures the total biomass attached to a surface, thus combining cells and biofilm matrix. In order to study whether agaric acid reduces the number of bacteria attaching to the surface, the number of CFU's in biofilms grown on the bottom of a glass petri dish was determined. This revealed that agaric acid also significantly inhibits the number of Salmonella cells attaching to the surface, although the inhibition was less strong than the biomass inhibition measured by crystal violet staining (FIG. 1C). Microscopic analysis confirmed that biofilms grown in presence of agaric acid are less dense compared to the control (FIG. 1D).

This inhibition was not due to a bactericidal effect as planktonic growth was not inhibited (FIG. 1B). Moreover, planktonic growth was enhanced at the highest concentration, indicating that agaric acid prevents attachment and results in more bacteria remaining in the planktonic phase. The minimal inhibitory concentration (MIC) of agaric acid under the same conditions as the biofilm assay was measured as 8 mM, further confirming that no bactericidal effects occurred at concentrations relevant for biofilm inhibition.

### Example 2. Agaric Acid Inhibits Flagellar Motility

To unravel the mechanism by which agaric acid inhibits biofilm formation, an in house developed reporter GFP-promoter fusion library was screened. This library contains reporters for 130 Salmonella genes related to biofilm formation, including genes regulating matrix production, fimbriae and flagella synthesis, quorum sensing and c-di-GMP regulation (Table 2) [Robijns et al. (2014). Biofouling 30, 605-625.]. A time-lapse of the first 24 h of biofilm formation in microtiter plates was performed to identify genes that are differentially transcribed in the presence of 100 μM agaric acid. As these reporter fusions express the stable GFPmut3 variant as a fluorophore, the measured fluorescence values are the accumulation of fluorescence over time [Robijns et al. (2014). Biofouling 30, 605-625](FIG. 2).

Remarkably, the transcription of central biofilm regulatory genes such as CsgD and RpoS was not downregulated in the presence of agaric acid. Additionally, the transcription of downstream genes such as csgB and adrA, respectively responsible for the production of curli fimbrae and cellulose, was not influenced by agaric acid [Steenackers et al. (2012) Food Research International 45, 502-531; Simm et al. (2014) Future microbiology 9, 1261-1282]. However, from 12 h onwards transcription of the flagellar sigma factor fliA is significantly inhibited by agaric acid. This downregulation was not caused by decreased transcription of f/hDC, the master regulator of motility in Salmonella [Das et al. (2018) Front Cell Infect Microbiol 8, 36-36], as the transcription of f/hDC is increased compared to the control between 9 h and 15 h. The repression of fliA significantly reduced the transcription of downstream class III flagellar gene flgM, flgK and motA, whereas transcription of tdcA flgB, fljB and fliC was not decreased at consecutive points. FlgM codes for an anti-sigma factor that directly binds and inhibits the transcription of class III genes. The FlgM protein is secreted by the flagellum-specific export apparatus, effectively coupling flagellar assembly with transcriptional regulation. FlgK is a hook-associated protein that stabilizes the hook-filament junction together with FlgL, whereas motor protein MotA is essential for driving the rotation of the flagella.

The reporter fusion data therefore suggest that agaric acid inhibits flagellar motility. Motility and biofilm formation are inversely regulated in Salmonella via the secondary signal molecule c-di-GMP. However, while the expression of genes necessary for flagellar motility is downregulated during biofilm maturation [Steenackers et al. (2012) Food Research International 45, 502-531], the initial adhesion on plastic or glass surfaces requires both flagella and active motility [Prouty and Gunn (2003) Infection and immunity 71, 7154-7158; Mireles et al. (2001) Journal of bacteriology 183, 5848-5854]. Inhibition of flagellar motility thus potentially explains the biofilm inhibitory effect of agaric acid.

To confirm that these changes in gene transcription also lead to phenotypic changes in motility, flagella of Salmonella grown in presence and absence of agaric acid were visualized by staining. However, no differences in flagellar appearance could be observed between the two conditions (FIG. 3A). A soft agar swimming assay was then performed to validate whether the downregulation of flagella genes in presence of agaric acid leads to reduced motility. In the absence of agaric acid, Salmonella formed a halo with an average diameter of 195 mm indicating migration from the start position via flagellar motility (FIG. 3B). Agaric acid completely abrogated this migration as no halo was formed, indicating a clear effect on the motility phenotype. Concurrently, a motA deletion mutant was also found to not show any migration, confirming that motA expression is necessary for flagellar motility under the conditions tested. These results indicate that agaric acid can completely inhibit motility, most likely not via inhibition of flagella synthesis but due to a downregulation of the flagellar motor protein MotA. In line with our hypothesis, deletion of motA inhibited biofilm formation to a similar extent as agaric acid. Importantly, addition of agaric did not further reduce biofilm formation in this deletion mutant, providing strong support for a mechanism where agaric acid reduces biofilm formation via inhibition of flagellar motility (FIG. 3C).

### Example 3. Salmonella Biofilms are More Sensitive to Antimicrobial Treatment in Presence of Agaric Acid

It is well established that biofilms can be extremely tolerant to treatment with antimicrobials. To test whether the preventive addition of agaric acid renders Salmonella biofilms more susceptible to treatment with common disinfectants or antibiotics, biofilms grown in the presence and absence of agaric acid for 48 hour were treated for 1 h with 0.25% H₂O₂ or 1 μM ciprofloxacin. Hydrogen peroxide is a commonly used disinfectant in food industry [Meireles et al. (2016) Food Research International 82, 71-85; Meireles et al. (2016) Food Research International 82, 71-85], whereas ciprofloxacin is a fluoroquinolone antibiotic frequently used to treat Salmonella infections [Tabak et al. (2009) FEMS microbiology letters 301, 69-76]. Biofilm formation has been shown to strongly protect Salmonella against either compound [González et al. (2018) Sci Rep 8, 222-222].

Biofilms grown in presence of agaric acid were significantly more susceptible to hydrogen peroxide. The increased sensitivity to treatment could be a consequence of the lower number of bacteria present, i.e. the inoculum effect Moreover, crystal violet staining already showed that agaric acid has a stronger inhibitory effect on the biofilm matrix than on the number of cells, indicating that the remaining attached cells are less protected by the matrix. Agaric Acid also further increased the effect of a ciprofloxacin treatment, albeit not significantly.

### Example 4. Agaric Acid has a Broad Spectrum Activity

It was tested whether agaric acid can also inhibit the biofilm formation of other opportunistic pathogens such as Pseudomonas aeruginosa, Staphylococcus aureus and Escherichia coli using the Calgary biofilm device. Agaric acid was found to inhibit biofilm formation of all three species. E. coli TG1 was even more sensitive to agaric acid than Salmonella as significant inhibition already occurred at 12.5 μM. Similarly to the case of Salmonella, planktonic growth was unaffected, except for an increase at the highest concentration. In contrast, agaric acid inhibits both the planktonic growth and biofilm formation of P. aeuruginosa PA14 and S. aureus SH1000. However, S. aureus biofilm inhibition occurred already at a lower concentration than the bactericidal effect, indicating that some biofilm-specific effects occur. Conversely, the main effect on P. aeuruginosa was bactericidal as planktonic growth is reduced at lower concentrations than biofilm inhibition. However, at high concentrations of agaric acid, biofilm formation is inhibited to a higher extent than planktonic growth. The flagellar systems of E. coli and Salmonella show a high degree similarity on the genetic and functional level [Albanna et al. (2018) Sci Rep 8, 16705]. Moreover, E. coli also requires normal flagellar function in order to successfully adhere to an abiotic surface [Pratt & Kolter (1998) Molecular microbiology 30, 285-293]. The specific biofilm inhibition of agaric acid on both Salmonella and E. coli thus further supports our hypothesis that agaric acid prevents biofilm formation via inhibition of flagellar rotation. Contrarily, S. aureus does not show flagellar motility, but rather moves via spreading or gliding [Pollitt & Diggle (2017) Cell Mol Life Sci 74, 2943-2958]. Therefore, agaric acid cannot inhibit S. aureus biofilm formation via interfering with the expression of genes responsible for flagellar rotation. Additionally, while Pseudomonas has flagella that are involved in adhesion and biofilm formation [O'Toole & Kolter (1998) Molecular microbiology 30, 295-304], the mainly bactericidal effect of agaric acid indicates that agaric acid has different targets in Pseudomonas. The mode of action of agaric acid is thus species dependent.

### Example 5. Agaric Acid Inhibits Biofilm Formation of Mixed-Species Communities

In situ, biofilms are typically complex communities consisting of various bacterial strains and species [Elias & Banin (2012) FEMS Microbiol Rev 36, 990-1004; Burmølle et al. (2014) Trends Microbiol 22, 84-91; Giaouris et al. (2015) Front Microbiol 6, Article 841; Mitri & Richard Foster (2013) Communities. Annual Review of Genetics 47, 247-273]. Since it is often reported that these bacterial consortia show enhanced biofilm formation compared to their monospecies counterparts it was determined whether agaric acid also inhibits the biofilm formation of mixed-species communities [Oliveira et al. (2015) PLoS Biol 13, e1002191-e1002191; Lories et al. (2020) Current Biology 30, 1231-1244; Burmølle, et al. (2006) Appl Environ Microbiol 72, 3916-3923; Ren et al. (2014). Microbial ecology 68, 146-154]. Hereto, a a previously characterized model community was utilized consisting of two Salmonella and one E. coli strain (S. Typhimurium SL1344 (S1), S. Typhimurium ATCC14028 (S2) and E. coli MG1655 (E1)). Agaric acid also significantly reduced the number of biofilm cells attached to the abiotic surface in this mixed-species consortium (FIG. 6A). The reduced biofilm productivity of the mixed-species community is mainly due to the inhibition of the S2 strain (FIG. 6B). The lack of 51 inhibition in mixed-species conditions is possibly due to agaric acid weakening the competitive interactions experienced by S1 (FIG. 6C). Previous research already showed that the presence of S2 and E1 significantly inhibits the growth of S1. Consequently, 51 detected the damage imposed by competition via its stress response systems and induces a competitive response. This competitive response included the upregulation of tolerance- and virulence-associated phenotypes and resulted in an enhanced tolerance of 51 towards antimicrobial treatment. Therefore, it was subsequently explored whether agaric acid can interfere with the increased tolerance in mixed-species conditions by weakening the competition and preventing the induction of this competitive response.

### Example 6. Agaric Acid Abolished the Enhanced Tolerance of S1 in Mixed-Species Conditions

It was confirmed that the presence of S2 and E1 increased the tolerance of 51 towards treatment with antimicrobials in the DMSO control (FIG. 7A). These finding are in line with prior reports showing that bacterial interactions within mixed-species biofilms can strongly enhance tolerance [Harriott & Noverr (2009). Antimicrob Agents Chemother 53, 3914-3922; Jahid et al. (2015). Food microbiology 46, 383-394; Parijs & Steenackers (2018) ISME J 12, 2061-2075; Schwering et al. (2013) Biofouling 29, 917-928; Lee et al. (2014). ISME J 8, 894-907]. As shown previously using a slightly modified set-up, preventive treatment with agaric acid reduced the tolerance of the remaining biofilm cells towards ciprofloxacin and H₂O₂. Moreover, S1 co-cultured with the other two strains no longer survived the antimicrobial treatment to a greater extent than the monoculture S1 biofilms. Agaric acid thus completely abolished the enhanced tolerance of S1 in mixed-species conditions. These results indicate that a reduction in competition prevented the competitive response and inhibited the increased tolerance associated with this response. However, weakening competition can initially increase the viability of unwanted bacteria. Therefore, it is thus crucial that the increased susceptibly towards antimicrobials sufficiently compensates for this initial increase in cell number and results in an absolute reduction of the remaining cells after treatment. Importantly, mixed-species communities in presence of agaric acid showed the lowest number of S1 cells surviving antimicrobial treatment (FIG. 7B). Agaric acid thus also shows strong potential to weaken mixed-species biofilm communities and reduce the tolerance towards antimicrobials.

### Example 7. Material & Methods

### Bacterial Strains and Culture Conditions

Overnight cultures (ONCs) of Salmonella enterica serovar Typhimurium ATCC14028, Escherichia coli TG1, Pseudomonas aeruginosa PA14, and Staphylococcus aureus SH1000 were grown at 37° C., shaken, with aeration, in Luria-Bertani (LB) broth, with 100 μg ml⁻¹ of ampicillin if appropriate.

### Antibiofilm Assay

A static peg assay for the prevention of bacterial biofilm formation was used as described previously. A lid carrying 96 polystyrene pegs was fitted into a microtiter plate with a peg hanging into each well. Two-fold serial dilutions of the compounds in 100 μl liquid broth per well were prepared in the microtiter plate. Subsequently, an overnight culture was diluted 1:100 into the respective liquid broth, and 100 μl (˜10⁶ cells) was added to each well of the microtiter plate, resulting in a total amount of 200 μl medium per well. After placing the lid on the microtiter plate, samples containing Salmonella, Pseudomonas or E. coli were incubated statically in TSB 1/20 for 48 h at 25° C., whereas S. aureus was incubated in undiluted TSB at 37° C. for 48 h. After incubation, the lid was removed from the microtiter plate and liquid culture was transferred to a new microtiter plate prior to determining the prior to determining the planktonic growth in each well via OD₆₀₀ measurements using a Synergy MX multimode reader (Biotek, Winooski, VT). The pegs were washed once in 200 μl PBS and the remaining attached bacteria were stained for 30 min with 200 μl 0.1% crystal violet in an isopropanol-methanol-PBS solution (1:1:18). Excess stain was washed off by placing the pegs in a 96-well plate filled with 200 μl distilled water per well. Afterwards, the pegs were air dried for 30 min and the dye bound to the adherent cells dissolved into 200 μl 30% glacial acetic acid. The OD₅₇₀ of each well was measured using a Synergy MX multimode reader. Data was analysed using the GraphPad Prism 6 software.

### Minimal Inhibitory Concentration (MIC) Assay

MIC values were determined in a 96-well plate. Two-fold serial dilutions of agaric acid or DMSO were prepared in 100 μl of TSB 1/20 and 100 μL of the inoculum diluted 1/100 in TSB 1/20 was added. The plate was covered with a Breathable Sealing Membrane and a lid and incubated for 24 h at 25° C., shaking at 200 rpm. The MIC was defined as lowest concentration of compound were Salmonella growth was lower than the upper bound of the 95% confidence interval of the negative control.

### Petri Dish Biofilm Assay

ONCs of S. Typhimurium ATCC 14028 were normalized to an OD₅₉₅ of 3.2 and diluted 1/100 in a small (60 mm Ø) glass Petri dishes containing 10 ml of 1/20 TSB to which a final concentration of 100 μM agaric or the corresponding amount of DMSO was added. Around 12*10⁷ ml⁻¹ cells were inoculated and incubated under static conditions at 25° C. for 48 h. Afterwards, the liquid above the biofilms was poured off and the biofilms were scraped off the bottom of the plate in 1 ml of PBS, passed through a syringe (25 G) and vortexed to break down the biofilm structure and ensure an homogenous suspension during dilution [Hermans et al. (2011). Journal of microbiological methods 84, 467-478]. The number of colony forming units (CFU) of biofilms was determined by plating.

### Microscopic Analysis

ONCs of S. Typhimurium ATCC 14028 containing the pFPV25.1 plasmid encoding for constitutive GFPmut3 productions were normalized to an an OD₅₉₅ of 3.2 and 20 μl was added to a glass bottom microwell dishes (35 mm Ø petri dish, 20 mm Ø microwell) containing 2 ml 1/20 TSB and 100 μg ml⁻¹ of ampicillin and a final concentration of 100 μM agaric or the corresponding amount of DMSO. Around 12*10⁷ ml⁻¹ cells were inoculated and incubated under static conditions at 25° C. for 48 h. After incubation, the planktonic phase was gently poured off and the biofilm was washed with 1 ml PBS. Biofilms were visualized with

### Microtiter-Plate-Based GFP Promoter Fusion Assay

1.5 μl of the reporter fusions' ONCs were transferred in three repeats to black polystyrene, clear-bottomed microtiter plates (Greiner bio-one 655096) containing 200 μl of 1/20 TSB with either a final concentration of 100 μM agaric or the corresponding amount of DMSO. Subsequently, the microtiter plates were incubated statically, at 25° C. for 24 h. Every 3 h, the fluorescence (excitation 488 nm, emission 511 nm) and absorbance at 600 nm (OD₆₀₀) were measured using a Synergy MX multimode reader. For data analysis, blank measurements were subtracted from both the fluorescence (using a promoterless pFPV25 vector as control) and OD₆₀₀. The ratio between the different OD₆₀₀ values of the strains/conditions was used to normalize any effects on the fluorescence caused by growth differences in the bacteria. Significant differences in the level of fluorescence between treatment and control were determined using a two-tailed Student t-test (P<0.05).

### Staining of Flagella

An ONC of Salmonella Typhimurium ATCC 14028 was diluted 1/100 in 5 ml TSB 1/20, with either a final concentration of 100 μM agaric or the corresponding amount of DMSO as a control. The planktonic cultures were incubated for 24 h at 25° C., shaking at 200 rpm. The flagella were stained according to Kearns and Losick (2003) Molecular microbiology 49, 581-590. Briefly, the stain consist of ten parts mordant (2 g tannic acid, 10 ml 5% phenol, 10 ml saturated aqueous AIK(SO₄)₂) mixed with one part stain (12% crystal violet in ethanol). 3 μl of sample was applied to a microscopic slide and covered with a 22 mm×40 mm coverslip. After placing the slide vertically, 10 μl was applied to the top edge of the coverslip in order to stain the sample due to capillary forces. Samples were visualized with phase contrast using a Zeiss Axio Imager Z1 microscope with an EC Plan Neofluar (×100 magnification/1.3 numerical aperture) objective.

### Soft Agar Swimming Assay

Based on Kim & Surette (2003) Biol Proced Online 5, 189-196, swimming plates were made by mixing 30 ml TSB 1/20 with 0.25% agar. These plates contained either a final concentration of 100 μM agaric or the corresponding amount of DMSO. After 2 h drying at room temperature, 3 μl of an overnight culture was inoculated by piercing the surface of the agar with the pipette tip. The plates were incubated upright for 24 h at 25° C., afterwards the size of the halo was measured and visually recorded.

### Tolerance Assay

To determine the tolerance of mature biofilms, biofilms were grown on microscopy glasses (75 mm×25 mm) placed vertical in a 50 ml falcon filled with 30 ml TSB 1/20. This set-up allows for easy transfer of mature biofilms as the top of vertical slide sticks out of the medium which allows to grab the slide with a pincer without damaging the biofilm. ONC of Salmonella were normalized to an OD600 of 3.2 and diluted 1/100 into the broth containing either a final concentration of 100 μM agaric or the corresponding amount of DMSO. After 48 h static incubation at 25° C., the glass slide was transferred to a new 50 ml falcon containing either 0.25% H₂O₂, 1 μM ciprofloxacin, or PBS and was incubated for 1 h. Afterwards, biofilms were scraped off the glass slide in 10 ml of PBS, passed through a syringe (25 G) and vortexed to break down the biofilm structure and ensure an homogenous suspension during dilution. The number of colony forming units (CFU) of biofilms was determined by plating.

### Mixed-Species Biofilm Assay

ONCs of S1, S2 and E1 were normalized to an OD₅₉₅ of 3.2 and added to a small (60 mm Ø) glass Petri dishes containing 10 ml of 1/20 TSB. The three strain were inoculated in a 1:1:1 ratio and incubated under static conditions at 25° C. for 48 h. The same total number of cells was inoculated in mono- and mixed-species conditions (˜12*10⁷ cells ml⁻¹). Afterwards, the liquid above the biofilms was poured off and the biofilms were scraped off the bottom of the plate in 1 ml of PBS, passed through a syringe (25 G) and vortexed to break down the biofilm structure and ensure an homogenous suspension during dilution. The number of colony forming units (CFU) of biofilms was determined by plating. To differentiate between the strains, S1 was labelled with constitutive gfpmut3 on a plasmid, while S2 and E1 were labelled with plasmid-encoded constitutive dsRed.T4. Differences in colony shape and size allowed differentiation between S2 and E1 during CFU counting.

### Mixed-Species Tolerance Assay

The monospecies and mixed-species biofilms were grown on petri dishes for 48 h at 25° C. as described above. The medium was then replaced by 5 ml PBS with either 0.25% H₂O₂ or 1 μM ciprofloxacin and the biofilms were incubated for an additional 1 h at 25° C. Subsequently, the biofilms were scraped off and plated out on solid LB agar plates for CFU determination. To differentiate between the strains, S1 was labelled with constitutive gfpmut3 on a plasmid, while S2 and E1 were labelled with plasmid-encoded constitutive dsRed.T4. Differences in colony shape and size allowed differentiation between S2 and E1 during CFU counting.

TABLE 2
Overview of all genes tested during GFP promoter fusion assay

| Gene | Function | Gene | Function |
|---|---|---|---|
| phoP | Biofilm regulators | pagC1 | Virulence |
| sinR | | hilA | |
| yhiW/STM3602 | | hilC | |
| ybaJ | | rstA | |
| csrA | | sirA/uvrY | |
| rpoS | | hha | |
| csgD | | invF | |
| mlrA/yehV | | ssaM | |
| katE | | sopA | |
| STM1987 | | sopB | |
| STM1437/ydhM | | pipB | |
| torS | | pipB2 | |
| crl | | sanA | Stress |
| csgB | Biofilm matrix | mig-14 | responses |
| adrA | production | cpxP | |
| bapA | | aadA | |
| bcsABZC | | STM4240 | |
| rcsA | | tolC | |
| rcsCDB | | yecG/STM1927 | |
| wcaA | | ompA | Metabolism |
| yihU | | fbp | |
| yihVW | | maeB | |
| srgE | Quorum sensing | pckA | |
| sdiA | | pps | |
| lsrA | | glpT | |
| pmrC/yjdB | | STM1939 | |
| fimA | Fimbrae | nadA | |
| lpfA | | purF | |
| pefA | | purH | |
| sthA | | purA | |
| flhD | Flagellar | guaB | |
| tdcA | motility | guaC | |
| flgB | | cyaA | |
| flgK | | carA | |
| flgM | | yijC | |
| fliA | | potF | |
| fliC | | sspA | |
| fljB | | mgtA | |
| motA | | kefC | |
| kefC | Metabolism | STM1255 | Other |
| nirB | | sciJ/STM0275 | |
| narK | | STM3071 | |
| oadG | | STM4423 | |
| dcoC/oadG2 | | ybeL | |
| ptsH | | STM0731 | |
| tig | | ydhO | |
| fur | | STM1851 | |
| nrdH | | yciG/STM1728 | |
| fhuA | | yjcO | |
| fes | | yciU/STM1740 | |
| iroN | | yegS/STM2140 | |
| sitA | | yeaH/STM1284 | |
| iroB | | STM0868 | |
| exbB | | ybdO | |
| ytfE | | | |
| hemA | | | |
| def | | | |
| aroF | | | |
| aroK | | | |
| aroQ/STM1269 | | | |
| thrA | | | |
| serA | | | |
| cysB | | | |
| cysD | | | |
| cysJ | | | |
| cysK | | | |
| hisB | | | |
| hisG | | | |
| ilvC | | | |
| asnA | | | |
| gltB | | | |
| metB | | | |
| leuA | | | |
| yeeF | | | |

1-11. (canceled)

12. An in vitro method of treating, decreasing formation, or delaying formation of a biofilm of micro-organisms on an abiotic surface, the method comprising:
contacting the abiotic surface with a carboxylic acid, wherein the carboxylic acid is a dicarboxylic acid or a tricarboxylic acid and has a chain length from 8 to 41 carbon atoms.

13. The method according to claim 12, wherein the micro-organisms are bacteria.

14. The method according to claim 12, wherein the carboxylic acid is a tricarboxylic acid.

15. The method according to claim 12, wherein the carboxylic acid is substituted with a hydroxyl group.

16. The method according to claim 12, wherein the carboxylic acid is a 1,2,3-tricarboxylic acid.

17. The method according to claim 12, wherein the carboxylic acid is a 2-hydroxy, 1,2,3-tricarboxylic acid.

18. The method according to claim 12, wherein the carboxylic acid has a chain length from 15 to 23 carbon atoms.

19. The method according to claim 12, wherein the carboxylic acid has a chain length from 17 to 21 carbon atoms.

20. The method according to claim 12, wherein the carboxylic acid is selected from the group consisting of 2-hydroxyheptadecane-1,2,3-tricarboxylic acid; 2-hydroxynonadecane-1,2,3-tricarboxylic acid; 2-hydroxyoctadecane-1,2,3-tricarboxylic acid; 2-hydroxy-4-oxononadecane-1,2,3-tricarboxylic acid, and 2-hydroxy-19-methylicosane-1,2,3-tricarboxylic acid.

21. The method according to claim 12, wherein the carboxylic acid is 2-hydroxynonadecane-1,2,3-tricarboxylic acid.

22. The method according to claim 12, wherein the carboxylic acid is contacted with said the abiotic surface at a concentration of at least 5 μM.

23. A method of treating a biofilm of microorganisms in an individual, the method comprising:
administering to the individual a carboxylic acid, wherein the carboxylic acid is a dicarboxylic acid or a tricarboxylic acid and has a chain length of from 8 to 41 carbon atoms.

24. The method according to claim 23, wherein the microorganisms are bacteria.

25. The method according to claim 23, wherein the carboxylic acid is administered by topical application of the carboxylic acid to the individual.

26. The method according to claim 23, wherein the carboxylic acid is a tricarboxylic acid.

27. The method according to claim 23, wherein the carboxylic acid is substituted with a hydroxyl group.

28. The method according to claim 23, wherein the carboxylic acid is a 1,2,3-tricarboxylic acid.

29. The method according to claim 23, wherein the carboxylic acid is a 2-hydroxy 1,2,3-tricarboxylic acid.

30. The method according to claim 23, wherein the carboxylic acid has a chain length of from 15 to 23 carbon atoms.

31. The method according to claim 23, wherein the carboxylic acid has a chain length of from 17 to 21 carbon atoms.

32. The method according to claim 23, wherein the carboxylic acid is selected from the group consisting of 2-hydroxyheptadecane-1,2,3-tricarboxylic acid; 2-hydroxynonadecane-1,2,3-tricarboxylic acid; 2-hydroxyoctadecane-1,2,3-tricarboxylic acid; 2-hydroxy-4-oxononadecane-1,2,3-tricarboxylic acid, and 2-hydroxy-19-methylicosane-1,2,3-tricarboxylic acid.

33. The method according to claim 23, wherein the carboxylic acid is 2-hydroxynonadecane-1,2,3-tricarboxylic acid.
